(19) United States
(12) Patent Application Publication
(10) Pub. No.: US 2007/0270938 A1
(43) Pub. Date: Nov. 22, 2007

(54) Device for Reinforcing and Relieving a Dilated Vessel

(76) Inventor: Peter Osypka, Rheinfelden-Herten (DE)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 11/574,097

(22) PCT Filed: Aug. 18, 2005
(86) PCT No.: PCT/EP05/08948

§ 371 Date: Feb. 22, 2007

(30) Foreign Application Priority Data
Aug. 26, 2004 (DE) 102004041259.6

(51) Int. Cl. A61F 2/82

(52) U.S. Cl. 623/1.12

(57) ABSTRACT

A device (1) for reinforcing and relieving a blood vessel (2) in a vessel dilation (3) thereof is provided. The device includes a stent (5) with metal reinforcement (4), which, in the position of use, bridges the vessel dilation (3) and can be anchored outside of the vessel dilation (3) on the blood vessel (2). The device (1) includes an insertion cannula (6), inside which the stent (5) is located during the insertion into the blood vessel (2) in a shape, in which it has a small radial extent. Once the stent (5) has left the insertion cannula (6), it automatically expands in order to correspond approximately to the vessel cross section. For anchoring the stent (5), the device is provided with pin anchors (7), which can pierce the vessel wall from inside, which are each attached to a traction element or thread (8), and which are set in a transverse arrangement after piercing through the vessel wall. The anchors (7) are connected to the stent (5) after it is positioned using the traction element (8).

### BACKGROUND

The invention relates to a device for reinforcing and relieving a blood vessel in the area of a vessel dilation (aneurysm), especially in an artery, with a stent, which features metal reinforcement, which bridges, in the position of use, the vessel dilation, and which can be anchored at least at an edge area outside of the vessel dilation, and with an insertion cannula, inside which the stent is located in the compressed position during the introduction into the blood vessel.

Despite great advances in aneurysm surgery, surgical removal of an abdominal aortic aneurysm or another type of aneurysm remains a considerable strain on the patient.

Therefore, interventional catheter techniques have been developed, which allow self-expanding stents to be placed in the abdominal area of the corresponding damaged artery. An example here is described in DE 28 05 749 A1.

Here, the seal between the vessel wall and the stent represents a large problem. For this purpose, the stent must be anchored to the vessel wall. This is done according to DE 28 05 749 A1 by means of anchoring pins, which are carried along with the stent expanding in its original position into the position of use, which simultaneously pierce the vessel wall due to the expanding motion, and which remain permanently in place in the position of use. Thus, these pin-like anchors must already be connected to the stent in advance, so that care must be taken during handling that these pin-like anchors are not damaged or do not change their position.

For this purpose, it is necessary that the stent is folded very exactly, so that it can hold each pin-like anchor in such folds. Inserting this folded stent is considered difficult especially when bends and changes in direction are necessary along the insertion path, wherein the folded outer side must be pushed along the vessel inner wall with the pin-like anchors located between the folds.

Self-expanding nitinol frames are also known as stents, which clamp to a certain extent in the interior of the blood vessel due to their expansion, leading to a permanent radial pressure against the vessel that is already damaged in some areas.

Furthermore, stents are known, which are fixed in the vessel wall by metal hooks. Here, the positioning and fixing is very difficult, because several hooks must be connected simultaneously to the vessel and repositioning is not possible.

This is also a concern for the arrangement according to DE 28 05 749 A1.

### SUMMARY

Therefore, there is the objective of creating a device of the type defined above, with a stent that can be positioned and anchored easily and securely.

To meet this objective, the device named above is characterized in that pin-like anchors, which can pierce through the vessel wall from the inside and which are each attached to a traction element or thread and can be passed through or pierced through the vessel wall by means of a tool or stiletto, are provided for anchoring, and in that the appropriate traction element or thread and therefore the appropriate anchor are connected or can be connected to the stent and/or to its reinforcement and/or to loops located in the stent and/or reinforcement.

In this way, it is possible to first position the stent carefully and to bring it into its position of use, without simultaneously also realizing the anchoring. Instead, this can be performed separate from the positioning procedure. Here, due to the traction elements or threads on the anchors, it is also possible to first bring the anchors onto the vessel wall in their relevant position and then to bring the stent along the thread into its correct position and then to connect to the threads. The anchoring and the positioning procedures are thus independent or “decoupled” from each other by the arrangement and construction of the device according to the invention.

Here it is considered especially advantageous that the threads provided on the pin-like anchors can be used as guidance aids for the stent to be positioned after attaching the anchor in the vessel wall, so that the position of the stent can also be defined in advance by the anchor to a large extent before its actual expansion and fixing.

Furthermore, the anchor can be moved completely through the vessel wall so that the wall is only pierced by the traction element or thread in the position of use, which produces a significantly better anchoring than a pin or hook sticking into the vessel wall.

Here, it is especially favorable when the stent can move relative to the traction elements or threads while being connected to these elements or threads. Therefore, the traction elements have a double function, in that they are used, on one hand, as a guide for the stent after the fixing of the pin-like anchor, after which they also allow their fixing in the desired position, for example, through tying at corresponding positions to the stent.

The appropriate anchor can have a formation as an attachment point for the tool or stiletto, which can be coupled to the tool detachably. Thus, the appropriate anchor can be gripped detachably with the help of this tool and pushed into its position of use.

Here, it is preferable when the anchor has, on its rearward side in the insertion direction, a reduced cross-sectional area for an insertable end of the tool and/or a receptacle opening that is open on the rearward end and that is closed on the opposite end for the rod-shaped tool or stiletto fitting into this opening, with which the anchor can be pierced into or through the vessel wall. Especially favorable is a pin-shaped or rod-shaped anchor with an opening, which is open towards the rearward end and closed towards its tip and in which the end of a tool or stiletto fits, so that it holds the anchor well and can have a corresponding non-positive or positive fit effect in the piercing or insertion direction. Nevertheless, after anchoring, the tool is accordingly easily withdrawn. Here, the anchor can preferably be pierced completely through the vessel wall, so that only the traction element connected to it runs back into the interior of the vessel from the anchor and is well anchored accordingly.

Here, it is especially preferred for good anchoring when the traction element attaches to the anchor between the two ends of the anchor. This allows the anchor to contact the outside of the vessel with its longitudinal side after the piercing by the anchor and the traction element or the thread to run approximately from the middle of the anchor back into the blood vessel. Such an attachment of the thread or traction element between the ends of the anchor simplifies the result that the anchor itself contacts the outside of the vessel over a surface area after reaching its position of use.

Attaching the anchor in its position of use can be considerably simplified when the device has an internal channel, in which the tool or stiletto can move, for the rod-shaped tool and when the inner cross section of this guide corresponds, in particular, approximately to the outer cross section of the anchor at least at its outer end and holds the anchor in the starting position at least partially within itself. The anchor can be inserted or possibly also placed at the end of the guide, through which the tool or stiletto is pushed, so that this tool can automatically grip the anchor and move it forward. Here, the anchor can be inserted or possibly also placed detachably on corresponding guides, in order to be brought together with the stent into its position of use, where they then are pressed out from the guide with the help of the stiletto and pierced through the vessel wall, which can be simplified by a tip at the end of each anchor.

Here, the device can have as many guides as anchors. The user can then grip the anchors held detachably by the individual guides with the help of a tool or stiletto one after the other and bring them into their anchored position, wherein the tool must be inserted gradually into the individual guides and must move in them sufficiently until it has reached the end of the guide and thus the appropriate pin-like anchor, which is automatically gripped by the tool and moved through the vessel wall for further motion.

A preferred construction of the invention of extremely advantageous significance is provided in that the device has an expanding body or cage, which can expand in the axial extent in the forward direction in front of the stent, which is permeable for blood, which can be pushed with or preferably in front of the stent out of the insertion cannula, which is arranged in front of the stent in the pushing direction, and which can be supported on the inside of the vessel due to the expandability or ability to spread, and in that the guides for the tool or tools for installing the anchors are arranged on this expanding body. Therefore, it is possible to fix the entire device within the vessel during the anchoring procedure, namely with the cage or expanding body, which automatically increases its cross section after leaving the insertion cannula or the insertion catheter, so that it contacts the vessel on the inside, whereby the guides carried by it simultaneously maintain their position of use. Therefore, it can be prevented that the forces appearing during anchoring lead to displacement of the guides for the other anchors, that is, the precision of the anchoring is simplified or improved primarily through this expandable or spreading body or cage.

The expandable body can have rods, which run approximately parallel to each other in the axial direction, which run together at their end regions, especially conically or diagonally, and the guides for the tool and the anchor are arranged approximately in the direction of these rods in the end region of the cage facing the stent and end in parallel profiles at or before the junction of these diagonal areas of the rods.

Such an expandable body made from parallel rods can be easily supported and form a contact on the inside of a vessel and is simultaneously permeable for the blood stream. Here, advantageously the transition of these parallel rods can be used at a middle starting point in the form of corresponding diagonal profiles of these rods, in order to hold the guides for the tool and the anchor in a favorable diagonal position, which then gives the corresponding direction for the insertion of the tool into the corresponding anchor and its displacement direction when piercing the vessel wall. Here, in an advantageous way the guides can open in the area of the diagonal bending in the axial region of the individual cage rods, so that the anchor can be handled unobstructed by the body or cage, which initially carries it.

The thread of the anchor can be arranged, in particular, loosely on the outside of the guides and can extend to the stent and the stent can be moved by means of the threads into its position of use and can be pushed out of the insertion cannula of the catheter and therefore expanded, wherein its ends on both sides of the vessel dilation can be pressed against the inside of the vessel wall. The vessel wall is initially held against restoring forces in the interior of the insertion cannula. When it leaves the insertion cannula, it automatically expands, so that the user only has to ensure that the stent is located in its position of use when it leaves the cannula. For example, he can bring the stent into the correct position with the help of the insertion cannula and then to hold it there initially out of the cannula and then to retract the cannula. It is also possible, however, to push the stent out of the position of the cannula retracted relative to the vessel dilation, that is, along the threads or traction elements of the anchor until the corresponding parts or regions of the stent, on which the traction elements or threads are attached initially only movably, have reached the anchors, where the traction elements can be fixed, preferably tied, after the final positioning and widening.

The inner cross section of the stent can have a size, through which the expandable body or cage can be retracted due to its elastic resilience. For example, this body or cage can be composed of nitinol, which has good flexibility. Thus, if the stent has reached its position of use and is also connected rigidly to the traction elements of the anchor in the axial direction, for example, tied, the expandable body can be retracted with the guides located on it and removed together with the insertion cannula.

Primarily through the combination of individual or several of the previously described features and measures, a device is produced, with which a stent can be placed and anchored easily in the region of an aneurysm, wherein high strength of the corresponding anchoring positions can be achieved, in that the pin-like anchors are pierced completely through the vessel wall and connected to the stent with traction elements, which they can also guide for placing the stent.

The insertion of the entire device in the area of the vessel dilation is also possible without a problem, because during this procedure the corresponding important parts, namely the stent itself, but also the inflatable or expandable body can be located within a correspondingly pliant or flexible insertion cannula, that is, it does not come into contact with the inside of the vessel.

It was already mentioned that the traction element or the thread, with which the corresponding anchor is connected, can be connected rigidly to the stent after reaching the position of use, for example, with the help of a tie. For this purpose, it is useful when another thread, with which the thread of the anchor can be tied, ends on the stent next to the traction element or thread of the anchor. For example, on one of the metallic reinforcements of the stent in an area near the end, a passage loop for the anchor thread and directly adjacent to this another loop as a holder for the other thread can be provided, so that both threads run directly one next to the other on the corresponding reinforcement of the stent and can be tied surgically in a known way.

### BRIEF DESCRIPTION OF THE DRAWINGS

Below, an embodiment of the invention is described in more detail with reference to the drawing. Shown in partially schematized representation are:

FIG. 1 is a view of a device according to the invention, in which a stent and a expandable or spreading body located in front of the stent in the advancing direction are arranged with guides for anchors still in an insertion cannula, which can be inserted and moved into the region of a vessel dilation,

FIG. 2 is an enlarged scale view of a front end of the stent in the advancing direction with traction elements, one of which leads to an anchor that is placed on a guide at the end and from there is pushed with the help of a stiletto and can be pierced through a vessel wall, wherein a second thread is arranged on the reinforcement of the stent, with which the anchor-thread can be tied after the anchoring and positioning of the stent,

FIG. 3 is a view according to FIG. 1 after the positioning of the expandable or spreading body in the advancing direction behind the vessel dilation, whereby simultaneously the guides with the anchors reach their position of use, wherein the cannula is retracted so far that it releases the expandable body but still contains the stent, and wherein the anchors are already brought into their position of use with the help of a tool or stiletto, that is, are pierced through the vessel wall from the inside to the outside and are then set transverse relative to the traction element or thread holding it,

FIG. 4 is an enlarged scale view in a longitudinal section of the end of the stent with its undulating reinforcement on the periphery and loops located on the end reinforcement for the traction elements, which run on the other reinforcements between these and the sleeve part of the stent,

FIG. 5 is a view of an anchor with its traction element,

FIG. 6 is a view of the attachment of a tool to the anchor shown in longitudinal section, wherein the traction element has slack in the direction opposite the insertion, wherein the tool runs through a guide located inside the device when the anchor is gripped according to FIG. 2,

FIG. 7 is a view according to FIGS. 1 and 3 after placing and expanding the stent, wherein the insertion cannula is retracted correspondingly but the spreading or expandable body is still arranged in a position farther in front relative to FIG. 3, and

FIG. 8 is a view of a part of the reinforcement of the stent with traction elements tied to loops provided there for fixing the stent in its anchored position shown in FIG. 7.

### DETAILED DESCRIPTION OF THE PREFERRED EMBODIMENTS

A device designated as a whole with 1 is used for reinforcing and relieving a blood vessel 2 in the area of a vessel dilation 3 (aneurysm), especially in an artery, for example, in the aorta.

The device 1 has a stent 5, which is provided with metal reinforcement 4, which bridges the vessel dilation 3 in the position of use (FIG. 7), and which, according to FIG. 7, can be anchored at an edge area outside of the vessel dilation 3 in a way still to be described.

The device 1 also includes an insertion cannula 6, inside which the stent 5 is located in the pressed together or optionally also folded position during the insertion into the blood vessel 2 and through the blood vessel up to the area of the vessel dilation 3. Specifically, within the insertion cannula 6, the stent 5 initially has a smaller radial extent than in the position of use, so that it can be brought with the help of the insertion cannula 6 through the corresponding blood vessel 2 to the position of use, that is, the dilation 3.

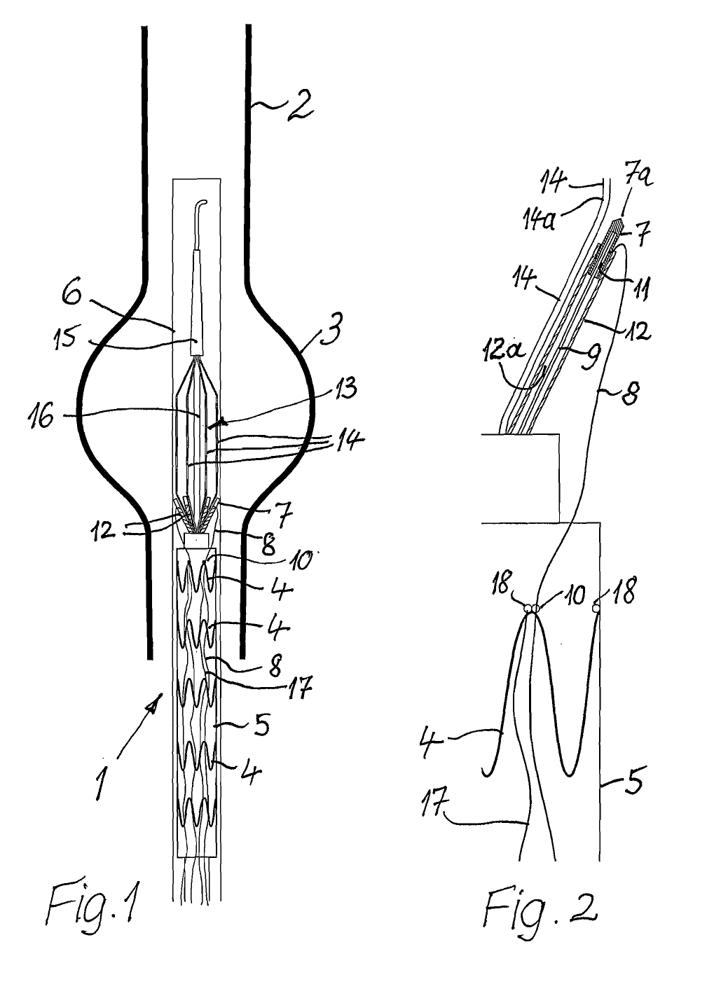

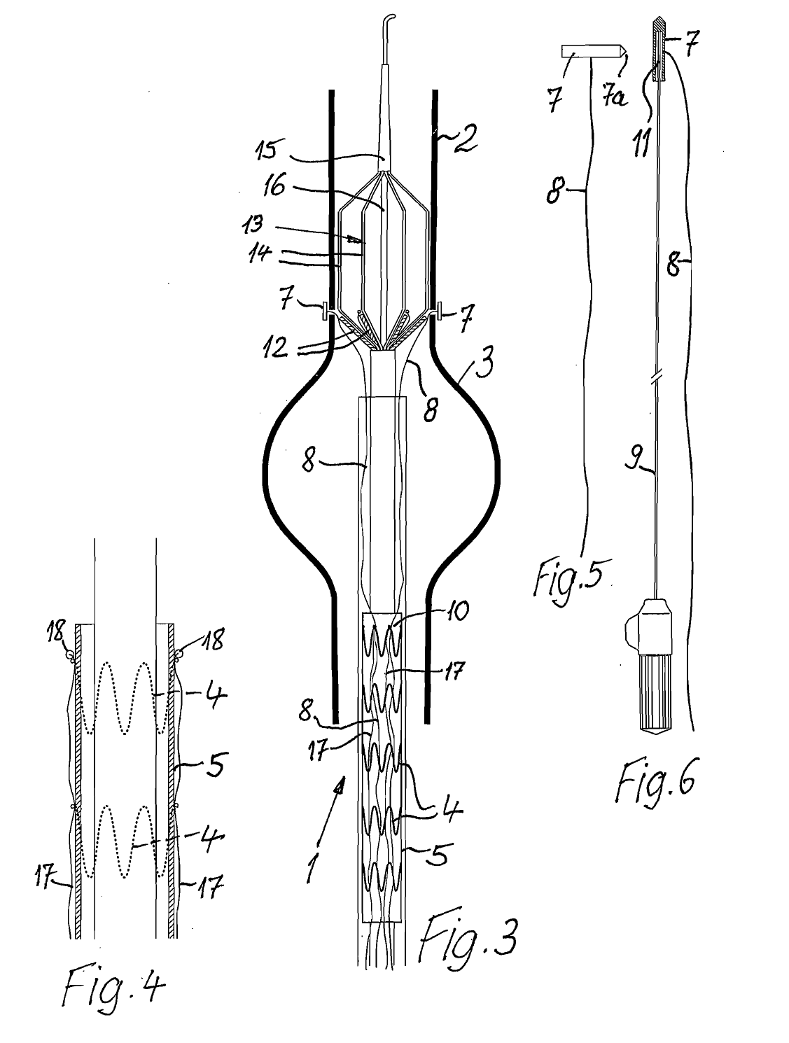

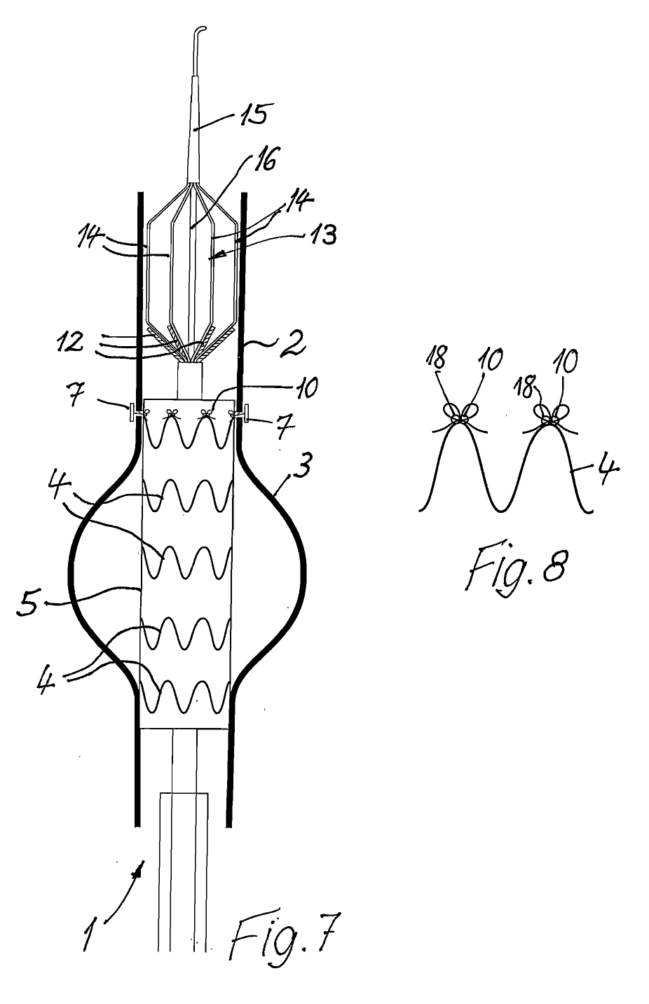

To anchor the stent 5 in its position of use, pin-like anchors 7 are provided, which can be pierced from the inside through the vessel wall according to FIGS. 3 and 7 and which are each attached to a traction element or thread 8 and can be pushed or pierced through the vessel wall by means of a tool or stiletto 9 (cf. FIGS. 2 and 6).

For this purpose, the pin-like anchors 7 have a tip 7a, which simplifies this piercing procedure, at their front end in the advancing direction.

According to FIGS. 2, 7, and 8, the corresponding element or the thread 8 and therefore also the corresponding anchor 7 are connected or can be connected to the stent 5 and simultaneously to its reinforcement 4 or to holders or loops 10 located on the stent 5 or on its reinforcement 4.

Here, with reference to FIGS. 1 to 4, one can see that the stent 5 is initially connected to the traction elements or threads 8 so that it can move relative to these elements or threads, that is, the threads 8 extend in a slipping way through the loops 10 located on the reinforcement 4, so that after setting the anchors 7, the stent 5 can be pushed along the thread 8 until it has reached its position of use. The stent 5 is thus displaceable at least initially relative to the traction elements or threads 8, but is simultaneously already connected to these elements or threads.

According to FIGS. 2 and 6, the anchor 7 has a formation, which can be coupled detachably to the tool 9 or its end, as an attachment position for the tool or stiletto 9. One can see that the anchor 7 has a receptacle opening 11 that is open on its rearward end in the direction of insertion and that is closed on the opposite end in the area of its tip 7a for the rod-shaped tool or stiletto 9 fitting into this opening 11, with which the anchor 7 can be pierced into or through the vessel wall. Thus, if the end of the stiletto 9 is pushed into the opening 11 and then the stiletto 9 in moved farther in the same direction, then it automatically carries along the anchor 7 until it has reached its position of use. Then the stiletto 9 can be retracted in a simple way, whereby the anchor remains in its position of use. In addition, the anchor is then set transverse relative to the traction element or thread 8, that is, it cannot slide back through the opening, which it created itself in the vessel wall.

So that the anchor 7 is automatically set transverse to the traction element or thread 8, the traction element 8 engages between the two ends of the anchor 7, in the embodiment approximately in the center. This transverse setting can be seen in FIGS. 3 and 7 and again at a larger scale in FIG. 5.

So that the tool 9 can grip and reach the individual anchors 7—one after the other—for each anchor 7 the device 1 has a guide 12, which has an inner channel 12a (cf. FIG. 2), in which the tool 9 or stiletto can be moved in the axial direction. Here, the inner cross section or the inner channel 12a of this guide 12 corresponds at least at the outer end approximately to the outer cross section of the anchor 7, so that the anchor 7 can be inserted partially into this guide 12 according to FIG. 2 and also according to FIG. 1, that is, approximately up to the center, where the traction element or the thread 8 separates. This is suspended on the outside on the guide 12 and runs directly to the outside of the stent 5 and the loop 10 provided there.

In FIGS. 1, 3, and 7 one can see that as many such guides 12 are provided here as anchors 7 for anchoring the stent 5, for example, four, five, six, or eight guides 12 for a corresponding number of anchors 7. One tool 9, however, is sufficient for the use of the individual guides 12 one after the other, in order to grip the anchors 7 held at the ends by a non-positive fit and to push them through the vessel wall.

According to FIGS. 1, 3, and 7, the device 1 has in the axial extent or in the advancing direction in front of the stent 5 an expandable or spreading body or cage 13, which is permeable for blood and which is initially arranged in the insertion cannula 6 (FIG. 1), and which can be pushed out of the insertion cannula 6 in front of the stent 5. This spreading body 13, which is permeable for blood due to its cage-like construction, is then supported on the inside of the vessel 2 in the position of use according to FIG. 3 due to its automatic expansion or spreading and thus fixes the important parts of the device 1 at least in advance. On this spreading body 13 namely the guides 12 for the tool or tools 9 for installing the anchors 7 are arranged. Thus, if this body or cage 13 is brought into the position shown in FIG. 3 and supported there, the anchors 7 can be positioned and fixed accordingly. Simultaneously, the threads 8 reach an end position, so that now the stent 5 can also be pushed out of the insertion cannula 6 relative to these threads 8, wherein simultaneously or thereafter the insertion cannula 6 is withdrawn until the position shown in FIG. 7 is attained.

The expandable body 13 has rods 14, which extend approximately parallel to each other in the axial direction and which extend together at their ends or end regions conically or diagonally, as can be seen especially well in FIGS. 3 and 7. Here, the ends of these diagonal regions facing each other are gripped by a central holder 15, from which a central rod 16 extends towards the insertion cannula 6 and thus holds and carries the expandable body 13 on both sides.

In the end area of the cage or body 13 facing the stent 5, the guides 12 for the tool 9 and the anchors 7 are arranged extending approximately in the diagonal direction of these rod areas on the diagonal rods of this body 13, which can be seen clearly in FIG. 2 but also well in FIGS. 1, 3, and 7. Here, these guides 12 end at or in front of the junction to the diagonal areas of the rods 14 in their parallel profiles. In FIG. 2, the curvature 14a of the initially diagonal rod 14 and thus the junction to the axial profile of this rod 14 can be identified and one sees that the anchor 7 is arranged with its tip 7a approximately at the height of this curvature 14a before it is pushed into its position of use. Thus, this displacement of the anchor 7 from the guide 12 can take place, without being obstructed by the rods 14.

Contributing to this is that the guide 12 is located on the outside of the expandable body or cage 13, that is, in FIG. 2 below the rod 14.

As already mentioned, the threads 8 of the anchors 7 are arranged loosely on the outside of the guides 12 and extend towards the stent 5 or to a loop 10 arranged on its reinforcement 4. Thus, the stent 5 can be moved by these threads 8 into its position of use and can be pushed out of the insertion cannula 6, wherein, conversely, the insertion cannula 6 can also be retracted relative to the stent 5. Thus, the automatically expandable stent 5 can expand automatically after leaving the insertion cannula 6 and receives the shape shown in FIG. 7, wherein its ends can be pressed onto the inside of the vessel wall on both sides of the vessel dilation 3, but without having to exert too great a pressure force, because the actual axial fixing is realized with the help of the anchor 7.

The inner cross section of the stent 5 has a size, through which the expandable body 13 can be retracted, also due to its elastic flexibility, because it can be composed of, for example, nitinol. FIG. 7 shows the body 13 shortly before its retraction, which can then bring it back into the retracted insertion cannula 6 together with the guides 12.

In FIGS. 2, 4 and also in FIG. 3 one can see that another thread 17 preferably ends and attaches on another loop 18, which is adjacent to the loop 10 and with which the thread 8 of the anchor 7 can be tied, on the stent 5 in addition to the corresponding traction element or thread 8 of the anchor 7. The tied arrangement is shown in FIG. 7 and enlarged in FIG. 8. After this tying of the threads 8 and 17, the stent 5 is finally fixed and anchored. Here, the attachment of the anchor 7 to one end is sufficient, because the blood stream is oriented so that it first enters into the end of the stent 5 with the anchor 7.

The metal reinforcement 4 on the outside of the stent 5 extends in an undulating arrangement on its periphery and is repeated in the axial direction. In FIG. 4 it is indicated that this reinforcement 4 runs on the outside of the stent 5, so that the threads 8 and 17 extend below the reinforcement 4, that is, between this and the outside of the stent 5, that is, guided from the reinforcements 4 and can be held in the radial direction.

The device 1 for reinforcing and relieving a blood vessel 2 in the area of a vessel dilation 3 includes a stent 5 with metal reinforcement 4, which, in the position of use, bridges the vessel dilation 3 and which can be anchored outside of the vessel dilation 3 on the blood vessel 2. This device 1 includes an insertion cannula 6, inside which the stent 5 is located during the insertion into the blood vessel 2 in a shape, in which it has a small radial extent, from which it can expand automatically after leaving the stent 5, in order to then correspond approximately to the vessel cross section.

To anchor the stent 5, there are pin-like anchors 7, which can be pierced from the inside through the vessel wall, which are each attached to a traction element or thread 8, and which are set in a transverse arrangement after piercing through the vessel wall. With the help of the traction element 8, the anchors 7 can be connected to the stent 5 after it is positioned.

In a preferred way, these anchor threads 8 can also support their guidance when moving the stent 5 into its position of use.

1. Device (1) for reinforcing and relieving a blood vessel (2) in an area of a vessel dilation (3) (aneurysm) comprising a stent (5), which has a metal reinforcement (4), which bridges the vessel dilation in a position of use, and which can be anchored on at least one edge area outside the vessel dilation (3), and with an insertion cannula (6), inside which the stent (5) is located in a position pressed together in a radial direction during insertion into the blood vessel (2), pin-like anchors (7) are provided for anchoring the stent, which can be pierced through a vessel wall from inside thereof, and are each attached to a traction element or thread (8), and can be pushed or pierced through the vessel wall by a tool or stiletto (9), and the corresponding traction element or the thread (8) and therefore the corresponding anchor (7) are connected or can be connected to the stent (5) and/or the reinforcement (4) or to holders or loops (10) located on the stent (5) or on the reinforcement (4).

2. Device according to claim 1, wherein the stent (5) is movable relative to the traction elements or threads (8) of the anchors (7) and is connected to the elements or threads.

3. Device according to claim 1, wherein or 2, the anchors (7) each have a formation, which can be coupled detachably to the tool (9), as an attachment point for the tool or stiletto (9).

4. Device according to one of claims 1, wherein the anchor (7) has on a rearward side in an insertion direction a reduced cross section area for an insertable end of the tool (9) and/or a receptacle opening (11) that is open at the rearward side and is closed at an opposite end into which the tool or stiletto (9) that is rod-shaped fits, with which the anchor (7) can be pierced in or through the vessel wall.

5. Device according to one of claims 1, wherein the traction element (8) is attached between two ends of the anchor (7).

6. Device according to claim 1, wherein a guide (12) for the rod shaped tool or stiletto (9) is provided, which has an inner channel (12a), in which the tool (9) or stiletto can be moved, and an inner cross section of this guide (12) corresponds on at least at an outer end thereof, approximately to an outer cross section of the anchor (7) and at least partially holds the anchor therein.

7. Device according to claim 6, wherein a plurality of guides (12) are provided equal to a number of the anchors (7).

8. Device according to claim 7, wherein an expandable body or cage (13) is located in an axial extent in an advancing direction in front of the stent (5), which is permeable for blood and which is pushable in front of or with the stent (5) out of the insertion cannula (6) and is supported on an inside of the vessel (2) due to an expandability or spreading thereof, and the guides for the tool or tools (9) for installing the anchors (7) are arranged on the expandable body (13).

9. Device according to claim 8, wherein the expandable body (13) has rods (14), which extend approximately parallel to each other in an axial direction and which extend together conically or diagonally at end areas thereof, especially, and the guides (12) for the tool (9) and the anchor (7) are arranged in end area of the cage (13) facing the stent (5) on the conical or diagonal end areas of the rods, extending approximately in a direction of the rods and end at or before a transition of the diagonal or conical end areas of the rods (14) into parallel profiles.

10. Device according to claim 7, wherein the threads (8) of the anchors (7) are arranged on an outside of the guides (12) and extend towards the stent (5) and the stent (5) can be moved into the position of use by the threads (8) and is pushable out of the insertion cannula (6) and therefore can be expanded, wherein ends of the stent can be pressed against an inside of the vessel wall on both sides of the vessel dilation (3).

11. Device according to claim 8, wherein an inner cross section of the stent (5) in the position of use has a size, through which the expandable body (13) can be retracted due to its elastic flexibility.

12. Device according to claim 1, wherein another thread (17), with which the thread (8) of the anchor (7) can be tied, ends at the stent (5) in addition to the traction element or thread (8) of the anchor (7).
